(19) United States
(12) Patent Application Publication
(10) Pub. No.: US 2006/0165644 A1
(43) Pub. Date: Jul. 27, 2006

(54) Cosmetics

(75) Inventors: Kazuaki Tanaka, Kanagawa (JP); Shoko Matsukuma, Kanagawa (JP); Tamie Suzuki, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: Fancl Corporation

(21) Appl. No.: 10/524,543

(22) PCT Filed: Aug. 14, 2002
(86) PCT No.: PCT/JP02/08271

§ 371 Date: Mar. 14, 2005

(51) Int. Cl. A61K 8/97; A61K 36/06; A61K 36/73; A61K 36/76; A61K 36/23

(52) U.S. Cl. 424/74; 424/195.17; 424/725; 424/195.16; 424/771; 424/757; 424/765

(57) ABSTRACT

Cosmetics containing humectants, cell activators, antiinflammatory agents, antioxidants and an Epilobium argustifolium extract, optionally together with jojova leaf, rosemary, extracts thereof and so on. These cosmetics contain C₄₋₇ diols. Antibacterial compositions containing orchid, Eriobotrya japonica, hop, rosemary, Filipendula multijuga, hawthorn, Persicae semen, Althaea officinalis, jojoba leaf, Epilobium argustifolium, extracts thereof and so on. Little irritative cosmetics characterized by being prepared by adding the antibacterial compositions to the above cosmetics.

### TECHNICAL FIELD

This invention relates to cosmetics, and more particularly to cosmetics, antibacterial compositions and cosmetics for sensitive skin which are possible to properly protect the human skin from external stresse, such as ultraviolet rays, and to repair the damaged skin.

### BACKGROUND ART

Generally, in the cosmetics, such as skin toners, beauty lotions, milky lotions, creams, packs and masks, cleansing agents, makeup cosmetics and so on, humectants such as glycerin, hyaluronic acid, plant extracts or so, cell activators such as placenta, royal jelly or so, anti-inflammatory agents such as glycyrrhizic acid derivatives, allantoin or so, anti-oxidants such as green tea extract, mugwort (Artemisia princeps) extact or so are blended according to desired effects such as skin care, whitening and the like.

On the other side, it is widely known that willowherb extract is an extract of willowherb (Epilobium angustifolium) in Onagraceae family of Onagraceae genus, and effective to alleviate erythema of the skin caused by applying ultraviolet rays or lactic acid.

Such the willowherb is a perennial herb not only distributed widely in the North America, but also growing gregariously in grasslands on plateaus of Hokkaido from north of the central part of Honshu, further of Asia, Europe and so on, and grows up to a height of 1.5 meters or so. Long and lanceolate leaves similar to those of willow alternate with each other in lengths of 5˜20 centimeters and have a tinge of glaucous white on the reverse sides thereof. Further it is known to put forth pretty, purplish red flowers in diameters of 1.3˜1.7 centimeters in a period from June to August.

Furthermore, oil (jojoba oil) extracted from seeds of jojoba (Buxus chinensis) has been used widely in an industrial field up to now, however studies have not been done substantially concerning leaves of jojoba. In recent years, the leaves of jojoba have been founded to be a plant effective for law material of the cosmetics.

In the cosmetics containing humectants, cell activators, anti-inflammatory agents or anti-oxidants independently, there is a problem in that desired effect can not be obtained sufficiently, and stability or usability as cosmetics is harmed in a case of blending the aforementioned agents in an amount sufficient to obtain the objective effect.

### DISCLOSURE OF THE INVENTION

The present invention has been made in view of the aforementioned problems, and it is an object to provide cosmetics, antibacterial compositions and cosmetics for sensitive skin, which are excellent in the stability and/or the usability, and superior in the skin care effect and/or the wrinkle care effect.

As a result of assiduously repeating investigations to solve the aforementioned problems, the inventors have found out that the aforementioned problems can be solved by using cosmetics containing willowherb and/or an extract therefrom, and humectants, cell activators, anti-inflammatory agents or anti-oxidants, independently or in combination, thereby completing this invention.

Further, the inventors have found out that the aforementioned problems can be solved by using cosmetics for sensitive skin composed of orchid, loquat (Eriobotrya japonica), hop (Humulus lupulus), rosemary (Rosmarinus officinalis), meadowsweet (Spiraea ulmaria), hawthorn (Cratoegus oxyacantha L.), peach kernel (Prunes persica Batsch), marsh mallow (Althaea officinalis), jojoba leaf, willowherb and extracts from these herbs, thereby accomplishing this invention.

Namely, the cosmetics according to this invention is characterized by containing willowherb and/or willowherb extract, and at least one selected from the group consisting of humectants, cell activators, anti-inflammatory agents and anti-oxidants.

The cosmetics according to an embodiment of this invention is characterized by further containing at least one selected from the group consisting of jojoba leaf, rosemary and extracts from these herbs.

Further, the cosmetics according to another embodiment of this invention is characterized in that the humectant is at least one selected from the group consisting of sweet hydrangea (Hydrangea macrophylla) leaf extract, carrot (Doucus carota) extract, marsh mallow extract, soybeans (Glycin soja) extract, Paeonia suffruticosa extract, coix seed (Coix lachryma-jobi) extract, angelica keiskei (Angelica keiskei) extract, rubus sauvissimus (Rubus sauvissimus) extract, yeast extract, bis-hydroxyethyl-bis-cetylmalonamide, sodium hyaluronate, L-serine, alanylglutamine, maltitol and rose hip oil.

Furthermore, the cosmetics according to the other embodiment of this invention is characterized in that the cell activator is at least one selected from the group consisting of brown algae extract, red algae extract, green algae extract, tocha (Ampelosis grossedentata) extract, aloe extract, sweet almond (Prunus amygdalus dulcis) extract, willow bark extract, jujube (Zizyphus jujuba) extract, natural vitamin E, corn glycogen, hydrolyzed yeast, rice bran oil, retinyl palmitate, palmitoyl pentapeptide, sea salt and sodium chloride.

Moreover, the cosmetics according to the other embodiment of this invention is characterized in that the anti-inflammatory agent is at least one selected from the group consisting of licorice (Glycyrrhiza glabra) extract, glycyrrhizic acid derivatives, glycyrrhetinic acid derivatives, burdock (Arctium lappa) extract, phellodendron (Phellodendri cortex) bark extract, lavender (Lavandula angustifolia) extract, peppermint (Mentha piperita) extract, zanthoxylum alatum alatum extract, calendula (Calendula officinalis) extract and panthenol.

Further, the cosmetics according to the other embodiment of this invention is characterized in that the anti-oxidant is at least one selected from the group consisting of jojoba leaf extract, green tea extract, chamomile (Chamomilla recutita) extract, soapberry (Sopindus mukurossi) peel extract, parsley extract, coptis rhizome (Coptis japonica) extract, mulberry (Morus bombycis) root extract, calendula extract, licorice extract and vitamin C.

Furthermore, the cosmetics according to the other embodiment of this invention is characterized by further containing diols having carbon atoms of 4˜7.

The antibacterial composition according to this invention is characterized by containing at least one selected from the group consisting of orchid, loquat, hop, rosemary, meadowsweet, hawthorn, peach kernel, marsh mallow, jojoba leaf, willowherb and extracts from these herbs.

Further, the cosmetics for sensitive skin according to this invention are characterized by adding the antibacterial composition into the aforementioned cosmetics.

Next, detailed explanation will be given concerning the cosmetics according to this invention. In this specification, “%” represents a mass percentage unless specified otherwise.

As mentioned above, the cosmetics according to this invention are composed by containing at least one selected from the group consisting of humectants, cell activators, anti-inflammatory agents and anti-oxidants, together with the willowherb extract. Accordingly, the skin care effect and the wrinkle care effect are raised as cosmetics, and the stability and the usability are improved.

Here, the following therapeutic agents may be listed concretely, as the above-mentioned humectants, cell activators, anti-inflammatory agents and anti-oxidants.

As the humectants, alkali simple spring water, deep seawater, yeast extract, mucopolysaccharides such as hyaluronic acid, sodium hyaluronate, chondroitin sulfate, dermatan sulfate, heparan sulffate, keratan sulfate and so, or derivatives of these mucopolysaccharides, proteins such as collagen, elastin, fibronectin, keratin and so, or derivatives, hydrolysis products and salts of these proteins, phospholipids such as phosphatidyl choline, phosphatidyl ethanolamine, phosphatidyl serine, phosphatidyl inositol, phosphatidyl glycerol, sphingomyelin and so, saccharides such as maltitol, xylitol, xylose, erythritol, pentaerythritol, fructose, dextrin and derivatives thereof, mannitol, sorbitol, inositol, trehalose, sucrose, glucose and so, seramide-like compositions such as glucocerebroside, galactocerebroside, lactosylceramide, seramide, bis-hydroxyethyl-bis-cetylmalonamide and so, urea, polyhydric alcohols such as glycerin, diglycerin, dipropyleneglycol and so, amino acids such as asparagine, aspartic acid, alanine, arginine, isoleucine, ornithin, glutamine, glycine, glutamic acid, cysteine, cystine, citrullin, threonine, serine, tyrosine, tryptophan, theanine, valin, histidine, hydroxylysine, hydroxyproline, phenylalanine, methionine, lysine, alanylglutamine, betaine and so, or derivatives of these amino acids, amino acid derivatives such as pyrrolidone carboxylic acid or salts thereof, plant extracts such as mucin, sweet hydrangea leaf extract, carrot extract, marsh mallow extract, soybeans extract, Paeonia suffruticosa extract, coix seed extract, angelica keiskei extract, rubus sauvissimus extract, fennel (Foeniculum vulgara) extract, peony (Paeonia albiflora) root extract and so, vegetable oils such as rose hip oil, sweet almond oil, perilla (Perilla frutescens) oil, olive (Olea europaea) oil, cacao butter, shea butter, borage oil, jojoba oil, macadamia (Macadamia ternifolia) nut oil, meadowfoam (Limnanthes alba) seed oil and so, animal oils such as squalan and so may be listed, for example.

It is especially preferable to use sweet hydrangea leaf extract, carrot extract, marsh mallow extract, soybeans extract, Paeonia suffruticosa extract, coix seed extract, angelica keiskei extract, rubus sauvissimus extract, yeast extract, bis-hydroxyethyl-bis-cetylmalonamide, sodium hyaluronate, L-serine, alanylglutamine, maltitol or rose hip oil, or to use them in optional combination.

As the cell activators, plant extracts such as brown algae extract, red algae extract, green algae extract, tocha extract, aloe extract, sweet almond extract, willow bark extract, jujube extract and so, vegetable oils such as rice bran oil and so, retinoid such as retinol, retinoic acid, retinyl palminate and so, vitamin E derivatives such as natural vitamin E, α-tocopherolum, vitamin E acetate and so, deoxyribonucleic acid and salts thereof, adenylic acid derivatives such as adenosine triphosphoric acid, adenosine diphosphoric acid, adenosine monophosphoric acid and so, salts of these derivatives, ribonucleic acid and salts thereof, cyclic AMP, cyclic GMP, flavin adenine nucleotide, guanine, adenine, cytosine, thymine, xanthine, and derivatives of these such as caffeine and theophylline, and nucleic acid related matters selected among salts of these, placenta extracts of pig, cattle and the like, calf blood extract, blood serum protein free extract, sleen extract, egg ingredients of hen and the like, cockscomb extract, fish meat extract, mollusk extracts such as sepia of cuttlefish, shell extract, shellfish extract, royal jelly, silk protein and decomposition products thereof, or derivatives of these, hemoglobin or decomposition products thereof, lactoferrin or decomposition products thereof, animal origin extracts such as mammalians, birds, fishes, mollusks, crustaceans, shell fishes, insects and so, microorganism origin extracts such as yeast extract, lactic bacterium extract, bifidus bacterium extract, ferment metabolite products and so, α and γ-linolenic acid, eicosapentaenoic acid and derivatives of these, hormones such as estradiol, ethenyl estradiol and so, organic acids such as glycolic acid, citric acid, lactic acid, malic acid, tartaric acid, salicylic acid, succinic acid and so, and derivatives and salts of these, corn glycogen, hydrolyzed yeast, palmitoyl pentapeptide, sea salt sodium chloride and so, may be listed, for example.

Especially, it is desirable to use brown algae extract, red algae extract, green algae extract, tocha extract, aloe extract, sweet almond extract, willow bark extract, jujube extract, natural vitamin E, corn glycogen, hydrolyzed yeast, rice bran oil, retinyl palmitate, palmitoyl pentapeptide, sea salt or sodium chloride, or to use them in optional combination.

Further, as the anti-inflammatory agents, plant extracts such as licorice extract, glycyrrhizic acid, glycyrrhizic acid derivatives, glycyrrhetinic acid, glycyrrhetinic acid derivatives, burdock extract, phellodendron bark extract, lavender extract, peppermint extract, zanthoxylum alatum alatum extract, calendula extract, lithospermi radix (Lithospermum officinale) extract, rose (Rosa multiflora) extract, bisabolol and so, allantoin, tranexamic acid, indometacin, azulene, ε--aminocapronic acid, hydrocortisone, hinokitiol, lysozyme chloride, diphenhydramine hydrochloride, panthenol and so, may be listed, for example.

Especially, it is preferable to use licorice extract, glycyrrhizic acid derivatives, glycyrrhetinic acid derivatives, burdock extract, phellodendri cortex bark extract, lavender extract, peppermint extract, zanthoxylum alatum alatum extract, calendula extract or panthenol, or to use them in optional combination.

Furthermore, as the anti-oxidants, plant extracts such as jojoba leaf extract, green tea extract, chamomile extract, soapberry peel extract, parsley extract, coptis rhizome extract, mulberry root extract, licorice extract, mugwort extact and so, L-ascorbic acid derivatives such as L-ascorbic acid, L-sodium ascorbic, L-ascorbyl-2-phesphate magnesium, ascorbyl glucoside, (ascorbyl/tocopheryl) potassium phosphate and so, dibutylhydroxytoluen, butylhydroxyanisole and so, may be listed, for example.

It is especially preferable to use jojoba leaf extract, green tea extract, chamomile extract, soapberry peel extract, parsley extract, coptis rhizome extract, mulberry root extract, calendula extract, licorice extract or vitamin C, or to use them in optional combination. The vitamin C is desirable to be ascorbyl glucoside, or (ascorbyl/tocopheryl) potassium phosphate, or optional combination thereof.

It is advisable to contain one or more of the aforementioned therapeutic agents (humectants, cell activators, anti-inflammatory agents and anti-oxidants) in a range of 0.0001˜20.0%, especially in a range of 0.01˜10.0% from the viewpoint of the stability, the usability and the skin care effect.

On the other side, the willowherb and/or the extract of willowherb have effects of abating the skin inflamed owing to the external stress such as ultraviolet rays and returning the skin to the natural state. For example, it is effective to lighten erythema caused by inflammation efficiently. Further, the willowherb and/or extract thereof alleviate irritation caused by high-functional ingredients.

The willowherb extract is obtained by extracting from the whole body of willowherb excepting the roots with various suitable solvents, which are not limited especially, after or without comminution in a dried or raw state, at room or elevated temperature, as typical preparation method.

As an extractant, for example, water, monohydric lower alcohols such as methanol, ethanol and so, liquid polyhydric alcohol as glycerin, propylene glycol, 1,3-butylene glycol and so, lower alkyl ether such as ethyl acetate and so, hydrocarbons such as benzene, hexane and so, ethers such as diethylether and so, animal and plant oils such as olive oil, jojoba oil, squalan and the like may be used independently or in proper combination thereof. Especially, it is advisable to use water, ethanol or 1,3-butylenes glycol, or mixture solvent composed by combining these extractants.

Further, content of the willowherb and/or extract thereof is desirable to be in a range of 0.00001˜5.0%, especially in a range of 0.001˜1.0% as dried solid matter from the viewpoint of the usability, skin care and wrinkle care effects.

Although it is known formerly that the willowherb extract functions as an anti-inflammatory component, the inventors have newly found out that the willowherb and/or willowherb extract give gives antibacterial properties. From the viewpoint of further improving the antibacterial properties, the cosmetics according to this invention is suitable to be contained with jojoba leaf, jojoba leaf extract, rosemary or rosemary extract, or optional combination thereof, whereby these therapeutic agents work as an auxiliary ingredient for the willowherb extract, and are effective for remarkably improving the antibacterial properties according to additive effects.

The jojoba leaf and the extract thereof have a function (SOD-type activity) for eliminating active oxygen, which is aging accelerant matter and generated by external stress such as ultraviolet rays, inhibit damage of cells and degeneration of collagen, and have a function for preventing the aging. They further exhibit tyrosinase-activity inhibition effect. It is advisable to contain the jojoba leaf and extract thereof in a proportion of 0.00001˜0.5%, especially in a range of 0.001˜0.15% as dried solid matter. The jojoba extract is obtained by extracting from leaves of Simmondisia cabinensis (Link) Schneider with water, ethanol, propylene glycol, 1,3-butylene glycol or mixed solution of these extractants.

The rosemary and the extract thereof have SOD-type activity, peroxidized fat inhibition effect, anti-inflammatory properties, melanin inhibition effect and antibacterial properties. They are desirable to be contained in a proportion of 0.00001˜0.2%, especially further preferable in a proportion of 0.001˜0.15% as dried solid matter. The rosemary extract is obtained typically by extracting from leaves or flowers of rosemary (Rosemarinus officianalis L.) with water, ethanol, propylene glycol, 1,3-butylene glycol, or mixed solution of these extractants, or ethanol solution contained with 1% of urea, or 1,3-butyleneglycol solution contained with 1% of urea.

Accordingly, by containing the aforementioned jojoba leaf rosemary or extract thereof, individual effects of these ingredients, namely, various effects such as anti-aging effect, whitening effect, anti-oxidizing effect, anti-inflammatory effect and anti-allergic effect can be given to the cosmetics.

Furthermore, in the cosmetics according to this invention, diols with carbon atoms of 4˜7 may be contained, thereby enabling storage for a long time, at the same time of further improving emollient effect for the skin and controlling degradation of various functions of this cosmetics (skin care effect, wrinkle care effect, anti-aging, anti-oxidizing effect, anti-inflammatory effect, anti-allergic effect and so on). As the diols having carbon atoms of 4˜7,1,2-pentanediol, 1,3-butylene glycol, dipropylene glycol, dipropylene glycol, isoprene glycol and so, may be used. Especially, 1,2-pentanediol and 1,3-butylene glycol can be suitably used, whereby antiseptics become unnecessary, and it is possible to ensure the high safety. The diols having carbon atoms of 4˜7 can be contained in a proportion of 0.1˜20 against the total mass of the cosmetics, especially it is preferable to contain in a range of 0.5˜10%.

Next, detailed explanation will be given about the antibacterial composition according to this invention.

Such the antibacterial composition is composed by containing orchid, loquat, hop, rosemary, meadowsweet, hawthorn, peach kernel, marsh mallow, jojoba leaf, willowherb or extracts of these herbs, or mixture composed of optional combination of these agents. It is especially desirable to contain rosemary, meadowsweet, hawthorn, jojoba leaf, willowherb or extracts of these herbs, or mixture composed of optional combination of these agents.

Antiseptics blended for purpose of only storage of long duration sometimes exhibit a strong irritating to the skin too much, or may bring about allergy, however antiseptic effect is produced by combining plant-originated materials in this invention, therefore it is possible to obtain remarkable high safety.

Therefore, cosmetics for sensitive skin can be obtained by adding the diols having carbon atoms of 4˜7 into the cosmetics, which is given with excellent antiseptic effect at the same time of ensuring various functions of this cosmetics (skin care effect, wrinkle care effect, anti-aging, whitening effect, anti-oxidizing effect, anti-inflammatory effect, anti-allergic effect and so on)

Next, explanation will be given about the characteristics of the respective ingredients possible to be used in the antibacterial composition according to this invention.

The orchid and the extract thereof have effects of moisturizing, inhibitation for fat peroxidization and so. The orchid extract is typically obtained by extracting from dried and comminuted roots of Epipactis Papillosa Franch ey Svat (Epipactis Papillosa Franch ey Savat), Neottia nidus-avis (L.) L. C. Rich. var mendshurica Komarov (Neottia nidus-avis (L.) L. C.Rich. var mendshurica Komer), Cephalalanthera longibracteata Blume (Cephalalanthera longibracteata Blume), variety of Ortis aristata Fisch (Dactylorhiaza incarnate (L.) Soo) and the like, with various suitable solvents (which are not limited especially), such as 1,3-butylene glycol or so, concretely. The orchid and the orchid extract are advisable to be contained in a proportion of 0.0001˜0.2%, especially preferable in a range of 0.001˜0.1% as dried solid matter, from the viewpoints of antibacterial properties and irritability to the skin.

The loquat and the extract thereof have effects of anti-allergic properties, anti-inflammatory properties, emollient properties, astringent properties, skin-softening properties and so. The loquat extract is obtained by extracting typically from leaves of the loquat (Eriobotrya japonica Lindley) with water, ethanol, 1,3-butylene glycol or mixed solution of these extractants, or dehydrated ethanol. The loquat and the loquat extract are desirable to be contained in a proportion of 0.0001˜0.2%, especially preferable in a range of 0.001˜0.1% as dried solid matter, from the viewpoints of antibacterial properties and irritability against the skin.

The hop and the extract thereof have effects such as antibacterial effect, analgesic effect, sedative effect, prevention effect for falling hair, anti-allergic effect and the like. The hop extract is typically obtained by extracting from the hop (Humulus lupulus L.) with water, propylene glycol, 1,3-butylene glycol or mixed solution of these extractants. The hop and the hop extract are advisable to be contained in a proportion of 0.0001˜0.2%, especially preferable in a range of 0.001˜0.1% as dried solid matter, from the viewpoints of antibacterial properties and irritability against the skin.

The rosemary and the extract thereof have effects of anti-oxidizing properties, antidandruff properties, anti-inflammatory properties, prevention for falling hair, inhibition for histamine liberation, antibacterial properties, astringency and so on and used also as spice. The rosemary extract is obtained typically by extracting from leaves and flowers of rosemary (Rosemarinus officianalis L.) with water, ethanol, propylene glycol, 1,3-butylene glycol, or mixed solution of these extractants, or ethanol solution contained with 1% of urea, or 1,3-butyleneglycol solution contained with 1% of urea. The rosemary and the rosemary extract are desirable to be contained in a proportion of 0.00001˜0.2%, especially preferable in a range of 0.0001˜0.15% as dried solid matter, from the viewpoints of antibacterial properties and irritability against the skin.

The meadowsweet and the extract thereof have effects of anti-inflammation, astringency, skin care and so. The meadowsweet extract is obtained typically by extracting from inflorescences or Fillipendula ulmaria Maximowicz (Fillipendula ulmaria Maximowicz) with propylene glycol, 1,3-butylene glycol, or mixed solution of these extractants. The meadowsweet and the meadowsweet extract are desirable to be contained in a proportion of 0.0001˜0.2%, especially preferable in a range of 0.001˜0.1% as dried solid matter, from the viewpoints of antibacterial properties and irritability against the skin.

The hawthorn and the extract thereof have effects of moisturizing, whitening, astringency and so on. The hawthorn extract is obtained typically by extracting from flowers, leaves, fruits and so of hawthorn (Cratoegus oxyacantha L.) with water, ethanol, propylene glycol, 1,3-butylene glycol, or mixed solution of these extractants. The hawthorn and the hawthorn extract are advisable to be contained in a proportion of 0.0001˜0.2%, especially preferable in a range of 0.001˜0.1% as dried solid matter, from the viewpoints of antibacterial properties and irritability against the skin.

The peach kernel and the extract thereof have effects of whitening, promotion of blood flow, anti-inflammation and so on. The peach kernel extract is obtained typically by extracting from seeds of peach (Prunes persica Batsch or Prunes persica Batsch var. davidiana Maximowicz) with water, ethanol, 1,3-butylene glycol, or mixed solution of these extractants. The peach kernel and the peach kernel extract are desirable to be contained in a proportion of 0.0001˜0.2%, especially preferable in a range of 0.001˜0.15% as dried solid matter, from the viewpoints of antibacterial properties and irritability against the skin.

The marsh mallow and the extract thereof have effects of softening of the skin, moisturizing, anti-inflammation and so on. The marsh mallow extract is typically obtained by extracting from roots, or roots and leaves of marsh mallow (Althea officinalis L.) with water, ethanol, propylene glycol, 1,3-butylene glycoll or 3-butyleneglycol solution contained with 1% of urea, or mixed solution of these extractants. The marsh mallow and the marsh mallow extract are desirable to be contained in a proportion of 0.0001˜0.2%, especially preferable in a range of 0.001˜0.1% as dried solid matter, from the viewpoints of antibacterial properties and irritability against the skin.

The willowherb and the extract thereof have effects of anti-inflammation and so on. The willowherb extract is obtained by extracting from the whole body excepting the roots of willowherb with water, ethanol, 1,3-butylene glycoll or mixed solution of these extractants. The willowherb and the willowherb extract are advisable to be contained in a proportion of 0.005˜2.0%, especially preferable in a range of 0.01˜1.0% as dried solid matter, from the viewpoints of antibacterial properties and irritability against the skin.

The jojoba leaf and the extract thereof have effects of SOD-type activity, anti-oxidizing properties and so on. The jojoba leaf extract is obtained by extracting from leaves of Simmondisia cbinensis (Link) Schneider with water, ethanol, propylene glycol, 1,3-butylene glycoll or mixed solution of these extractants. The jojoba leaf and the jojoba leaf extract are desirable to be contained in a proportion of 0.0001˜0.2%, especially preferable in a range of 0.0001˜0.15% as dried solid matter, from the viewpoints of antibacterial properties and irritability against the skin.

In the cosmetics according to this invention, it is possible to blend ingredients commonly used for pharmaceutical preparation of cosmetics, quasi-drugs, drugs for external use and so, such as water, medicinal oils, surface active agents, gelling agents, thickeners, pulverulent bodies, ultraviolet absorbents, aromatics, essential oils such as jasmine oil, orange oil and so, synthetic or natural coloring agents, antiseptics, pH regulators, various therapeutic agents and the like in an amount of ordinary use besides the above-mentioned ingredients within a range of not impairing the effects of his invention.

Furthermore, the present cosmetics can be formed into various configurations known as cosmetics by mixing together with according to the ordinary method. Although the cosmetics according to this invention are possible to be made, for example, into basal cosmetics such as milky lotions, creams, skin toners, packs and masks. oils and so, face cleansing cosmetics and body shampoos, hair care cosmetics such as shampoos, rinses, hair treatments, hair creams, hair sprays, hair tonics, hair conditioners, hair growth promoters and so, makeup cosmetics such as foundations, face powders, lipsticks, mascaras, eye shadows, eye liners, eye brows, manicure preparations and so, external use drugs such as axillary deodorants, ointments, suspensions and so on, this invention is not limited to the aforementioned configurations. Moreover, the cosmetics according to this invention are not restricted also in the dosage form, and possible to appropriately form in various shapes such as solid type, paste type, mousse type, gel type, powder type, solution type, solubilization type, emulsification type, powder dispersion type, multilayer type and so on.

### BEST MODE FOR CARRYING OUT THE INVENTION

Next, the present invention will be further explained in detail with reference to examples, however this invention is not limited to these examples.

### INVENTIVE EXAMPLES 1˜3 AND COMPARATIVE EXAMPLES 1, 2

The cosmetics were prepared as precriptions shown in table 1 according to the ordinary method, and the obtained cosmetics were evaluated concerning the usability, skin care effect, the wrinkle care effect, the antiseptic property, the stability, and the safety. The respective properties were evaluated by the following method.

Usability (Stickiness, Skin Roughness)

Each of 10 sensory panelists evaluated a feeling at the time of applying proper quantities of respective samples to the skin.
◯: A case where 8 or more out of 10 panelists evaluated the sample to be insensible of sticky and skin roughness feelings.
Δ: A case where 5˜7 out of 10 panelists evaluated the sample to be insensible of sticky and skin roughness feelings.
×: A case where 4 or below out of 10 panelists evaluated the sample to be insensible of sticky and skin roughness feelings.
   Skin Care Effect, Wrinkle Care Effect

Ten woman panelists of 20˜49 years applied the sample to their face twice a day (morning, evening) for a month, and evaluated skin care effect and wrinkle care effect.
◯: A case where 8 or more out of 10 panelists evaluated the sample to be effective.
Δ: A case where 5˜7 out of 10 panelists evaluated the sample to be effective.
×: A case where 4 or below out of 10 panelists evaluated the sample to be effective.
   Antiseptic Properties

After inoculating microorganisms suspension to the sample of 20 g, a change of the number of bacteria was examined through the plate count mrthod. As the inoculated bacteria, Pseudomonoas aeruginosa, Escherichia coli, Staphylococcus aureus were used. The antiseptic properties of the sample were evaluated according to the change of the number of bacteria until the lapse of 4 weeks, the obtained results were classified in conformity to the following standards of three ranks.
⊚: A case where remarkable reduction of bacteria was observed.
◯: A case where reduction of bacteria was observed.
×: A case where reduction of bacteria was hardly observed or not observed entirely.
   Stability

After charging the obtained sample into a glass bottle and leaving it for a month at a room temperature, the sample was evaluated as to presence of separation, turbidity and decrease of viscosity by macroscopic observation.

Safety

The sample was applied to each of 20 woman subjects (35˜55 years) having sensitive skins easy to cause trouble in their skins against cosmetics, chemical substance and change of the external environment, twice a day (morning, evening) for a week continuously, and irritability to the skin (itching, stinging) was evaluated.

TABLE 1

| | Inventive | | | Comparative | |
|---|---|---|---|---|---|
| | Example | | | Example | |
| | 1 | 2 | 3 | 1 | 2 |
| Glycerin | 2 | 2 | 2 | 2 | 2 |
| 1,3-butylene glycol | 5 | 5 | 5 | 5 | 5 |
| 1,2-penthanediol | 0.5 | 0.5 | 0.5 | 0.5 | 0.5 |
| Polyoxyethylenesorbitan ester | 0.1 | 0.1 | 0.1 | 0.1 | 0.1 |
| of fatty acid | | | | | |
| Willowherb extract | 0.05 | 0.05 | 0.05 | 0 | 0 |
| (dried solid matter) | | | | | |
| Jojoba leaf extract | 0.01 | 0 | 0.01 | 0 | 0 |
| (dried solid matter) | | | | | |
| Rosemary extract | 0.01 | 0.01 | 0 | 0.01 | 0 |
| (dried solid matter) | | | | | |
| Parsley extract | 0.1 | 0.1 | 0.1 | 0 | 0.1 |
| Sodium hyaluronate | 0.1 | 0.1 | 0.1 | 0 | 0.1 |
| Willow bark extract | 3 | 3 | 3 | 0 | 3 |
| Glycyrrhizic acid derivatives | 0.1 | 0.1 | 0.1 | 0 | 0.1 |
| Purified water | Bal. | Bal. | Bal. | Bal. | Bal. |
| Usability | ◯ | ◯ | ◯ | X | Δ |
| Skin care effect, | ◯ | ◯ | ◯ | X | Δ |
| Wrinkle care effect | | | | | |
| Antiseptic properties | ⊚ | ⊚ | ◯ | ◯ | X |

In any cosmetics according to inventive examples 1˜3, the separation, the turbidity and the decrease of viscosity were not observed and they were recognized to be stable. Further, there was no person to complain of the irritability to the skin (itching, stinging).

Next, examples of prescriptions of skin toner, toner, cream, milky lotion, W/O foundation and face cleansing cosmetics are shown as inventive examples 4˜9, respectively.

(Inventive example 4: skin toner)

| | |
|---|---|
| Glycerin | 2.0% |
| Martitol | 1.0% |
| 1,3-butylene glycol | 5.0% |
| 1,2-penthanediol | 1.0% |
| Polyoxyethylenesorbitan ester of fatty acid | 1.0% |
| Willowherb extract | 5.0% |
| Green tea extract | 0.1% |
| Phellodendron bark extract | 0.1% |
| Brown algae extract | 0.1% |
| L-serine | 0.01% |
| Chamomile extract | 0.1% |
| Willow bark extract | 3.0% |
| Rosemary extract | 0.1% |
| Glycyrrhizic acid derivatives | 0.1% |
| Geranium oil | proper dose |
| Purified water | balance |

The skin toner of inventive example 4 was prepared by the above-mentioned prescription according to the ordinary method.

(Inventive example 5: toner)

| | |
|---|---|
| Sorbitol | 4.0% |
| 1,3-butylene glycol | 8.0% |
| Jojoba leaf extract | 0.05% |
| 1,2-penthanediol | 0.5% |
| Polyoxyethylene hardened castor oil | 0.5% |
| Willowherb extract | 3.0% |
| Alanylglutamine | 0.01% |
| Calendula extract | 0.1% |
| Paeonia suffruticosa extract | 0.1% |
| Hydrolyzed yeast | 0.1% |
| Aloe extract | 5.0% |
| Yeast extract | 1.0% |
| Rosemary extract | 0.1% |
| Parsley extract | 0.1% |
| Jasmine oil | proper dose |
| Potassium dihydrogen phosphate | proper dose |
| Purified water | balance |

The toner of inventive example 5 was prepared by the above-mentioned prescription according to the ordinary method.

(Inventive example 6: cream)

| | |
|---|---|
| Liquid paraffine | 10.0% |
| Beeswax | 3.0% |
| Cetanol | 2.0% |
| Stearic acid | 1.0% |
| Olive oil | 1.0% |
| Polyglycerin ester of fatty acid | 2.0% |
| Glycerin ester of fatty acid | 0.5% |
| Sorbitol | 3.0% |
| Dipropylene glycol | 5.0% |
| 1,2-penthanediol | 2.0% |
| Willowherb extract | 5.0% |
| Xanthan gum | 0.1% |
| Potassium hydroxide | 0.05% |
| Coix seed extract | 0.1% |
| Green algae extract | 0.1% |
| Retinyl palmitate | 0.1% |
| Mulberry root extract | 0.1% |
| Bis-hydroxyethyl-bis-cetylmalonamide | 0.1% |
| Ascorbyl glucoside | 5.0% |
| Licorice extract | 0.5% |
| Meadowsweet | 0.1% |
| Lavender oil | proper dose |
| Purified water | balance |

The cream of inventive example 6 was prepared by the above-mentioned prescription according to the ordinary method.

(Inventive example 7: milky lotion)

| | |
|---|---|
| Glycerin | 3.0% |
| 1,2-penthanediol | 0.5% |
| 1,3-butylene glycol | 3.0% |
| Marsh mallow extract | 1.0% |
| Hops extract | 1.0% |
| Orchid extract | 1.0% |
| Behenyl alcohol | 0.9% |
| Stearic acid | 1.0% |
| Glyceryl monostearate | 1.4% |
| Polyoxyethylenesorbitan monostearate (E.O.) | 0.8% |
| Squalane | 2.0% |
| Sweet hydrangea leaf extract | 1.0% |
| Willowherb extract | 2.0% |
| Tocha extract | 0.5% |
| Burdock extract | 0.1% |
| Coptis rhizome extract | 0.1% |
| Purified water | balance |

(Inventive example 8: W/O foundation)

| | |
|---|---|
| Dimethylsiloxane | 10.0% |
| Isononyl isononanate | 5.0% |
| Behenyl alcohol | 4.5% |
| Glycyrrhetic acid derivatives | 0.01% |
| Anhydrous silicic acid | 7.0% |
| Iron oxide | 1.5% |
| Titanium oxide | 9.0% |
| Talc | 2.5% |
| Polyethel-modified silicone | 3.0% |
| Willowherb extract | 1.0% |
| Sodium hyaluronate | 0.001% |
| Natural vitamin E | 0.01% |
| Glycerin | 5.0% |
| Jojoba leaf extract | 0.1% |
| Rice bran oil | 0.01% |
| Hawthorn | 0.1% |
| Orange oil | proper dose |
| Purified water | balance |

The W/O foundation of inventive example 8 was prepared by the above-mentioned prescription according to the ordinary method.

(Inventive example 9: face cleansing liquid)

| | |
|---|---|
| Deep seawater | 4.0% |
| Triethanolamine N-acyl gultamate (30% solution) | 5.0% |
| Lauric diethanolamide | 5.0% |
| Willowherb extract | 1.0% |
| Glycyrrhizic acid derivatives | 0.1% |
| Hydroxyethylcellulose | 0.1% |
| Peach kernel extract | 1.0% |
| Loquat extract | 1.0% |
| Rose hip oil | proper dose |
| Purified water | balance |

The face cleansing liquid of inventive example 9 was prepared by the above-mentioned prescription according to the ordinary method.

As a result of performing evaluation concerning the cosmetics obtained in the above-mentioned inventive examples 4˜9 in the same manner as the examples 1˜3, every one of these cosmetics was confirmed to be excellent in the stability and the antiseptic property. The sticky and skin roughness feelings were not conscious and the skin care effect and/or wrinkle care effect was recognized. Furthermore, there was no person to complain of the irritability to the skin (itching, stinging).

Additionally, in the inventive examples 4˜9, actual blending amounts of the extracts obtained in the market were described as the amounts of respective “extracts” in the above-mentioned prescriptions, therefore there is no description of the amounts of the extracts blended as dried solid matter.

### INVENTIVE EXAMPLE 10

### Antibacterial Plant Extract Fluid

By using orchid extract (ORCHID Liquid made by ICHIMARU PHARCOS LTD), loquat leaf extract (BIWA leaf Liquid B made by ICHIMARU PHARCOS LTD), hops extract (HOPS EXTRACT BG made by MARUZEN PHARMACEUTICALS CO. LTD), rosemary extract (ROSEMARY EXTRACT BG-J made by MARUZEN PHARMACEUTICALS CO. LTD), meadowsweet extract (made by KOEI PERFUMERY CO. LTD), hawthorn extract (CRATAEGUS Liquid B made by ICHIMARU PHARCOS LTD), peach kernel extract (TOUNIN Liquid B made by ICHIMARU PHARCOS LTD), marsh mallow extract (ORGANIC ALTHEA EXTRACT BG-50 made by KOEI PERFUMERY CO. LTD), jojoba leaf extract (JOJOBA LEAF EXTRACT made by KOEI PERFUMERY CO. LTD), and willowherb extract (2/133100 Willowherb Extract made by DRAGOCO JAPAN LTD), antibacterial activity of the respective ingredients and effect for reducing inoculated bacteria in samples selected randomly were examined.

Measurement of Antibacterial Activity

The antibacterial activity of these plant extract fluid was measured by using the micro-liquid culture method.

Further, applied concentration of samples and samples to be subjected to the medium test were prepared sequentially by using 8 steps double diluted solution of purified water. Pseudomonas aeruginosa and Candida albicans were pre-cultivated in the SCD bouillon medium and the GP medium and collected respectively, the obtained microorganisms suspension were inoculated together with the samples after diluting Pseudomonas aeruginosa and Candida albicans with sensitive bouillon and the GP medium, respectively. Pseudomonas aeruginosa and Candida albicans were cultivated at 37° C. and 25° C., respectively and a growth inhibition rate of the well applied with samples to the non-sample well was calculated in the respective concentration, thereby obtaining 50% growth inhibition concentration (converted concentration into solid matter) of the respective plant extracts against the microbes. The obtained results are shown in Table 2.

TABLE 2

| | IC50 against | IC50 against |
|---|---|---|
| | Pseudomonas | Candida |
| | aeruginosa (%) | Albicans (%) |
| Orchid extract | 0.03 | 0.02 |
| Loquat leaf extract | 0.03 | 0.06 |
| Hops extract | 0.03 | 0.09 |
| Rosemary extract | 0.07 | 0.1 |
| Meadowsweet extract | 0.03 | 0.04 |
| Hawthorn extract | 0.06 | 0.05 |
| Peach kernel extract | 0.06 | 0.1 |
| Marsh mallow extract | 0.03 | 0.05 |
| Jojoba leaf extract | 0.02 | 0.1 |
| Willowherb extract | 0.07 | 0.12 |

Evaluation of Reduction Effect for Inoculated Bacteria

Pseudomonas aeruginosa, Escherichia coli, Staphylococcus aureus of about 10⁷ were inoculated to the samples shown in Table 3 per 1 g of the respective samples. The viable microbe cell number were measured by plate count method after cultivation for 7 days at 25° C.

The viable microbe cell number on seventh day was calculated as a residual rate by defining the number of inoculated microbes to 100%, and the antibacterial activity of the samples was evaluated by classifying the residual rate into standards of four ranks as follow. The results are shown in Table 3.

⊚; Residual rate lower than 0.01%

◯; Residual rate lower than 10%, not lower than 0.01%

Δ; Residual rate lower than 100%, not lower than 10%

×; Residual rate higher than 100%

TABLE 3

| Sample | Sample | Antibacterial |
|---|---|---|
| No. | (Concentration converted into solid matter) | activity |
| 1 | Rosemary extract 0.05% | ◯ |
| 2 | Jojoba leaf extract 0.05% | ◯ |
| 3 | Willowherb extract 0.25% | ◯ |
| 4 | Willowherb extract 0.125% | ◯ |
| | Jojoba leaf extract 0.025% | |
| 5 | Willowherb extract 0.125% | ⊚ |
| | Jojoba leaf extract 0.025%, BG 5% | |
| 6 | Willowherb extract 0.125% | ⊚ |
| | Jojoba leaf extract 0.025% | |
| | Rosemary extract 0.125% | |
| 7 | Willowherb extract 0.125% | ⊚ |
| | 1,3-butylene glycol 5% | |
| 8 | Willowherb extract 0.125% | ⊚ |
| | 1,2-pentanediol 0.5% | |
| 9 | 1,3-butylene glycol 10% | ◯ |
| 10 | 1,2-pentanediol 1.5% | ◯ |

It is clear from Table 2 that the respective ingredients to be contained in the antibacterial composition according to this invention exhibit the excellent antibacterial activity against both Pseudomonas aeruginosa and Candida albicans.

Furthermore, as is apparent from Table 3, these active ingredients exhibit further excellent antibacterial activity by combining to use with each other. It is obvious that the antibacterial activity is remarkably improved in a case of blending dials having 4˜7 carbon atoms such as 1,2-pentanediol and 1,3-butylene glycol into willowherb extract and jojoba leaf extract. Moreover, the antibacterial activity becomes evident to be improved remarkably by arbitrarily combining willowherb extract, rosemary extract and jojoba leaf extract as compared with the case of using these extracts individually.

### INDUSTRIAL APPLICABILITY

As explained above, according to this invention, it is possible to provide the cosmetics, antibacterial composition and cosmetics for sensitive skin excellent in the stability and usability, and having the improved skin care effect and/or wrinkle care effect, because of applying the cosmetics containing willowherb and/or the extract thereof, and humectants, cell activators, anti-inflammatory agents or anti-oxidants, or optional combination of these agents, or applying the cosmetics for sensitive skin added with the antibacterial composition containing orchid, loquat, hop, rosemary, meadowsweet, hawthorn, peach kernel, marsh mallow, jojoba leaf, willowherb, extracts from these herbs or so.

1. Cosmetics containing willowherb and/or an extract therefrom, and at least one selected from the group consisting of a humectant, a cell activator, an anti-inflammatory agent and an anti-oxidant.

2. The cosmetics as set forth in claim 1, which further contain at least one selected from the group consisting of jojoba leaf, rosemary and extracts from these herbs.

3. The cosmetics as set forth in claim 1, wherein said humectant is at least one selected from the group consisting of sweet hydrangea leaf extract, carrot extract, marsh mallow extract, soybeans extract, Paeonia suffruicosa extract, coix seed extract, angelica keiskei extract, rubus sauvissimus extract, yeast extract, bis-hydroxyethyl-bis-cetylmalon-amide, sodium hyaluronate, L-serine, alanylglutamine, maltitol and rose hip oil.

4. The cosmetics as set forth in claim 1, wherein said cell activator is at least one selected from the group consisting of brown algae extract, red algae extract, green algae extract, tocha extract, aloe extract, sweet almond extract, willow bark extract, jujube extract, natural vitamin E, corn glycogen, hydrolyzed yeast, rice bran oil, retinyl palmitate, palmitoyl pentapeptide, sea salt and sodium chloride.

5. The cosmetics as set forth in claim 1, wherein said anti-inflammatory agent is at least one selected from the group consisting of licorice extract, glycyrrhizic acid derivatives, glycyrrhetinic acid derivatives, burdock extract, phellodendron bark extract, lavender extract, peppermint extract, zanthoxylum alatum alatum extract, calendula extract and panthenol.

6. The cosmetics as set forth in claim 1, wherein said anti-oxidant is at least one selected from the group consisting of jojoba leaf extract, green tea extract, chamomile extract, soapberry peel extract, parsley extract, coptis rhizome extract, mulberry root extract, calendula extract, licorice extract and vitamin C.

7. The cosmetics as set forth in claim 1, which further contain diols having carbon atoms of 4˜7.

8. An antibacterial composition containing at least one selected from the group consisting of orchid, loquat, hop, rosemary, meadowsweet, hawthorn, peach kernel, marsh mallow, jojoba leaf, willowherb and extracts from these herbs.

9. Cosmetics for sensitive skin containing the antibacterial composition as set forth in claim 8 in cosmetics containing willowherb and/or an extract therefrom, and at least one selected from the group consisting of a humectant, a cell activator, an anti-inflammatory agent and an anti-oxidant.

10. The cosmetics as set forth in claim 2, wherein said humectant is at least one selected from the group consisting of sweet hydrangea leaf extract, carrot extract, marsh mallow extract, soybeans extract, Paeonia suffruicosa extract, coix seed extract, angelica keiskei extract, rubus sauvissimus extract, yeast extract, bis-hydroxyethyl-bis-cetylmalon-amide, sodium hyaluronate, L-serine, alanylglutamine, maltitol and rose hip oil.

11. The cosmetics as set forth in any one of claim 2, wherein said cell activator is at least one selected from the group consisting of brown algae extract, red algae extract, green algae extract, tocha extract, aloe extract, sweet almond extract, willow bark extract, jujube extract, natural vitamin E, corn glycogen, hydrolyzed yeast, rice bran oil, retinyl palmitate, palmitoyl pentapeptide, sea salt and sodium chloride.

12. The cosmetics as set forth in any one of claim 3, wherein said cell activator is at least one selected from the group consisting of brown algae extract, red algae extract, green algae extract, tocha extract, aloe extract, sweet almond extract, willow bark extract, jujube extract, natural vitamin E, corn glycogen, hydrolyzed yeast, rice bran oil, retinyl palmitate, palmitoyl pentapeptide, sea salt and sodium chloride.

13. The cosmetics as set forth in any one of claim 2, wherein said anti-inflammatory agent is at least one selected from the group consisting of licorice extract, glycyrrhizic acid derivatives, glycyrrhetinic acid derivatives, burdock extract, phellodendron bark extract, lavender extract, peppermint extract, zanthoxylum alatum alatum extract, calendula extract and panthenol.

14. The cosmetics as set forth in any one of claim 3, wherein said anti-inflammatory agent is at least one selected from the group consisting of licorice extract, glycyrrhizic acid derivatives, glycyrrhetinic acid derivatives, burdock extract, phellodendron bark extract, lavender extract, peppermint extract, zanthoxylum alatum alatum extract, calendula extract and panthenol.

15. The cosmetics as set forth in any one of claim 4, wherein said anti-inflammatory agent is at least one selected from the group consisting of licorice extract, glycyrrhizic acid derivatives, glycyrrhetinic acid derivatives, burdock extract, phellodendron bark extract, lavender extract, peppermint extract, zanthoxylum alatum alatum extract, calendula extract and panthenol.

16. The cosmetics as set forth in any one of claim 2, wherein said anti-oxidant is at least one selected from the group consisting of jojoba leaf extract, green tea extract, chamomile extract, soapberry peel extract, parsley extract, coptis rhizome extract, mulberry root extract, calendula extract, licorice extract and vitamin C.

17. The cosmetics as set forth in any one of claim 3, wherein said anti-oxidant is at least one selected from the group consisting of jojoba leaf extract, green tea extract, chamomile extract, soapberry peel extract, parsley extract, coptis rhizome extract, mulberry root extract, calendula extract, licorice extract and vitamin C.

18. The cosmetics as set forth in any one of claim 4, wherein said anti-oxidant is at least one selected from the group consisting of jojoba leaf extract, green tea extract, chamomile extract, soapberry peel extract, parsley extract, coptis rhizome extract, mulberry root extract, calendula extract, licorice extract and vitamin C.

19. The cosmetics as set forth in any one of claim 5, wherein said anti-oxidant is at least one selected from the group consisting of jojoba leaf extract, green tea extract, chamomile extract, soapberry peel extract, parsley extract, coptis rhizome extract, mulberry root extract, calendula extract, licorice extract and vitamin C.

20. The cosmetics as set forth in any one of claim 2, which further contain diols having carbon atoms of 4˜7.
